SEQUENCE LISTING (10 sequences)

SEQ ID NO: 1 - protein, 349 aa, Yarrowia lipolytica
MVLSTVIGEWFSRVLFGTVAPSPLTATAPISQDFYDTALTFSHLSNVAYCINTPLESLKSDFSCGVACSHFPNMELVEEFGGEFFETSITGFLSIDHVKKEKYVVYRGTYDIGDVYTDIQLSQSPFLVTPSALGSTANLCEGCTIHDGWNKAYNETMGIIGDKLADHVNSNPDYRLVVTGHSLGAAIAVLSATSLKVNGQDPYLYTYGQPRIGNANFANFVSKQWFGEGDGLSMDSDRRYFRLTHWNDLFVGFPAFKDYVHSVGEIYIDYFTVQPPLNKVFSCAGPESMSCYRKDFNALARLDIVKNHLAYFDWISLCTLNIGRRDLERGRKFEGTWLYGGLANGSTIF

SEQ ID NO: 2 - protein, 307 aa, Yarrowia lipolytica
AVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNDHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 3 - protein, 347 aa, Yarrowia lipolytica
QTAPITQETYDFVLKYGWLSNVAYCVRAPGPFALQSDFTCGNSCAHFPDVTLDYQFGGNFFSTSVTGFLAHDHTKKEKYIVFRGTFSIADAITDIQTIQQPYMTSIPPLNTTDINSTNPSASINCPGCQVHDGFQKAYRETMVNVQDRLVDFLGNNTDYKLIVTGHSLGAVTALFMGINLKNLGYDPTLINYGQPRLGNKAFADYVDALFFKQGDDGLTINPERRMYRVTHWNDFFVGWPAGYSHTLGEVYISDPTGINAPIEDVYSCAGPENNQCHHGSFNLLERLNILKNHCGYLNWIFYCAINVDKREMMIDPPRVGKRVEHWSGKFSDVESTEGLMYEAIYPM

SEQ ID NO: 4 - protein, 366 aa, Yarrowia lipolytica
MVSFGARIKDFFSVLLFGAASTSSSTKTALVSQGFYDAALDFSHLSNIAYCVNAPITPLKSDFSCGQSCVHFPDIELVHIFGGDFFSTSITGYLALDHVKKEKYVVFRGTFSIADAITDIQFQQSSFLVNVPALNTFTANDTAPEAQIDCKQCKIHDGFSKAFTETWHNIGDLLEQHLDSYPDYQLYVTGHSLGAAMALLGATSIKLRGYDPILINYGQPRVGNKAFADYISALWFGNGDGLEINQQRRLYRMTHWNDVFVGLPNWDGYTHSNGEVYIKGKYVNPPLKDVFSCAGGENSKCYRSEFNLLAQINLLQNHLCYIDYIGFCALNVGRRELNDLPHYNGPYKYGHKTEEQFIAEGLELSN

SEQ ID NO: 5 - DNA, 1047 bp, Yarrowia lipolytica
  source 1..1047: mol_type = genomic DNA
atggttttgtctactgttattggtgaatggttctctagagttttgtttggtactgttgctccatctcctttgactgctactgctccaatctctcaagatttctacgatactgctttgactttttctcatttgtctaacgttgcttactgtatcaacactccattggaatctttgaagtctgatttctcttgtggtgttgcttgttctcactttcctaacatggagttggttgaagagttcggtggtgaatttttcgagacttctattactggtttcttgtctatcgatcatgttaagaaagagaagtacgttgtttacagaggtacttacgatatcggagatgtttacactgatatccaattgtctcaatctccattcttggttactccttctgctttgggttctactgctaatttgtgtgaaggttgtactattcacgatggttggaacaaggcttataatgagactatgggtattattggagataaattggctgatcatgttaactctaatcctgattacagattggttgttactggtcactctttgggtgctgctattgctgttttgtctgctacttctttgaaggttaacggtcaagatccatacttgtatacttacggtcaacctagaatcggtaacgctaacttcgctaacttcgtttctaagcaatggtttggtgaaggagatggtttgtctatggattctgatagaagatacttcagattgactcattggaacgatttgtttgttggtttcccagcttttaaggattacgttcactctgttggtgaaatctacatcgattacttcactgttcaaccacctttgaacaaggttttctcttgtgctggtcctgagtctatgtcttgttacagaaaggatttcaacgctttggctagattggatatcgttaaaaatcatttggcttacttcgattggatttctttgtgtactttgaacattggtagaagagatttggaaagaggtagaaagtttgagggtacttggttgtatggtggtttggctaatggttctactattttc

SEQ ID NO: 6 - DNA, 921 bp, Yarrowia lipolytica
  source 1..921: mol_type = genomic DNA
gctgttttgcaaaagagagtttacacttctactgaaacttctcatatcgatcaagaatcttacaactttttcgagaagtatgctagattggctaatattggttattgtgttggtccaggtactaagatttttaagcctttcaactgtggtttgcaatgtgctcatttcccaaacgttgaattgatcgaagagtttcacgatcctagattgattttcgatgtttctggttacttggctgttgatcatgcttctaagcaaatctacttggttattagaggtactcactctttggaggatgttatcactgatatcagaatcatgcaagctccattgactaactttgatttggctgctaatatttcttctactgctacttgtgatgattgtttggttcacaacggtttcattcaatcttacaacaacacttacaatcaaattggtccaaagttggattctgttattgagcaataccctgattatcaaattgctgttactggtcattctttgggtggtgctgctgctttgttgttcggtattaacttgaaggttaatgatcacgatccattggttgttactttgggtcaacctattgttggtaacgctggtttcgctaattgggttgataagttgtttttcggtcaagaaaacccagatgtttctaaggtttctaaggatagaaagttgtacagaatcactcatagaggagatattgttccacaagttcctttttgggatggttatcaacattgttctggagaggttttcattgattggcctttgattcacccacctttgtctaatgttgttatgtgtcaaggtcaatctaacaaacaatgttctgctggtaacactttgttgcaacaagttaacgttattggtaatcacttgcaatacttcgttactgaaggtgtttgtggtatt

SEQ ID NO: 7 - DNA, 1041 bp, Yarrowia lipolytica
  source 1..1041: mol_type = genomic DNA
caaactgctccaatcactcaagaaacttacgatttcgttttgaagtacggttggttgtctaacgttgcttactgtgttagagctccaggtccttttgctttgcaatctgatttcacttgtggtaactcttgtgctcatttccctgatgttactttggattaccaattcggtggtaactttttctctacttctgttactggtttcttggctcacgatcacactaagaaagaaaagtacatcgtttttagaggtactttctctatcgctgatgctattactgatatccaaactatccaacaaccatacatgacttctattccacctttgaacactactgatatcaactctactaatccatctgcttctattaattgtcctggttgtcaagttcacgatggttttcaaaaagcttacagagagactatggttaacgttcaagatagattggttgatttcttgggtaacaacactgattacaagttgatcgttactggtcactctttgggtgctgttactgctttgtttatgggtattaacttgaaaaatttgggttacgatccaactttgattaactatggtcaacctagattgggtaataaggctttcgctgattacgttgatgctttgtttttcaaacaaggagatgatggtttgactattaacccagaaagaagaatgtacagagttactcattggaacgatttctttgttggttggcctgctggttactctcacactttgggtgaagtttatatttctgacccaactggtattaacgctcctattgaagatgtttactcttgtgctggtccagagaacaatcaatgtcatcacggttcttttaatttgttggaaagattgaacatcttgaagaatcattgtggttacttgaactggattttctattgtgctattaatgttgataagagagaaatgatgattgatccacctagagttggtaaaagagttgagcactggtctggtaaattttctgatgttgaatctactgagggtttgatgtacgaggctatctaccctatg

SEQ ID NO: 8 - DNA, 1047 bp, Yarrowia lipolytica
  source 1..1047: mol_type = genomic DNA
atggtcctgtccactgttatcggagaatggttttcaagagttttgtttggtactgtcgccccatcaccactcaccgccacagcccccatttctcaggacttctacgataccgctctcacgttctctcacctatctaatgttgcttactgtatcaacacccctcttgagtccctcaagtccgacttttcctgcggtgtcgcatgttctcacttcccaaacatggaacttgttgaagagttcggtggagaatttttcgaaacttctattactggttttctatctatcgaccatgtcaaaaaggaaaagtacgttgtgtacagaggcacctatgacattggtgacgtttatactgacattcaattaagccaatccccattcttggttaccccttctgccctgggttctacagcaaatctgtgtgagggttgtactatccacgatggttggaacaaagcatacaacgaaaccatgggtattatcggagacaagctcgctgaccacgttaactccaacccagattacagattggtcgtaactggacattcacttggtgctgctattgctgtactatctgcaacctctcttaaggtcaatggccaagatccttacctttacacttacggtcagcctcgtattggtaatgctaacttcgcaaactttgtgagtaagcaatggttcggtgagggtgacggtctatctatggactcggacagacgttatttcagactgactcactggaacgacctctttgttggtttccctgctttcaaggactacgtgcactctgtcggagagatttacatagactatttcaccgttcaaccaccactcaacaaagttttctcttgtgctgggcctgagtctatgtcctgttacagaaaggacttcaacgcccttgctagacttgatattgtgaaaaaccacctagcttacttcgattggatctctctgtgcaccctaaacatcggcagacgtgatctggagaggggtagaaagttcgagggtacttggctctacggtggactggctaacggatccactatcttc

SEQ ID NO: 9 - protein, 339 aa, Rhizomucor miehei
VPIKRQSNSTVDSLPPLIPSRTSAPSSSPSTTDPEAPAMSRNGPLPSDVETKYGMALNATSYPDSVVQAMSIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 10 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

(19) United States
(12) Patent Application Publication
(10) Pub. No.: US 2024/0334947 A1
(43) Pub. Date: Oct. 10, 2024

(54) YARROWIA LIPOLYTICA AND ITS USE FOR PRODUCING LIPASES SPECIFICALLY LIBERATING SHORT CHAIN FATTY ACIDS

(71) Applicant: Chr. Hansen A/S, Hoersholm (DK)

(72) Inventors: Christian Jaeckel, Hoersholm (DK); Lone Riisberg, Hoersholm (DK)

(73) Assignee: Chr. Hansen A/S, Hoersholm (DK)

(21) Appl. No.: 18/688,225

(22) PCT Filed: Aug. 31, 2022
(86) PCT No.: PCT/EP2022/074190

§ 371 Date: Feb. 29, 2024

(30) Foreign Application Priority Data
Sep. 1, 2021 (EP) 21194329.5

(51) Int. Cl. A23C 19/032; A23C 13/16; C12N 1/16; C12N 9/20; C12R 1/645

(52) U.S. Cl. CPC A23C 19/0328; A23C 13/16; A23C 19/0325; C12N 1/165; C12N 9/20; C12Y 301/01003; C12R 2001/645

(57) ABSTRACT

The present invention relates to a Yarrowia strain, preferably a non-genetically modified Yarrowia strain, for the production of a wild-type, non-engineered, microbial lipolytic enzyme with a high specificity for short-chain fatty acids, preferably with a higher specificity for short chain fatty acids than for medium to long chain fatty acids, preferably when compared to a known microbial lipolytic enzyme. This invention is suitable for use in the food industry, preferably in the dairy industry, more preferably in the cheese industry leading, for example, to flavor enhancement or shortening of the ripening times for ripened cheeses.

### TECHNICAL FIELD

The present invention relates to Yarrowia as a host for the production of a wild-type, non-engineered, microbial lipolytic enzyme with a high specificity for short-chain fatty acids, preferably with a higher specificity for short chain fatty acids than for medium to long chain fatty acids, preferably when compared to a known microbial lipolytic enzyme. Preferably, the invention relates to Yarrowia lipolytica as a host for the production of a homologous microbial lipolytic enzyme with a high specificity for short-chain fatty acids. Preferably, the Yarrowia strain is a non-genetically modified strain, more preferably the Yarrowia lipolytic strain is a non-genetically modified strain.

This invention is suitable for use in the food industry, preferably in the dairy industry, more preferably in the cheese industry leading, for example, to flavor enhancement or shortening of the ripening times for ripened cheeses.

### BACKGROUND

Lipolytic enzyme also called lipases (EC 3.1.1.3) are a class of hydrolases that hydrolyze ester bonds of lipids of triglycerides generating free fatty acids. This class of enzymes is responsible, for example, for the lipolysis of milk fat and/or other fat and subsequent release of short (C₄- to C₆-fatty acids), medium and/or long (C₈- and higher) chain fatty acids.

Each lipolytic enzyme has its own release profile of free fatty acids from milk fat and/or other fat. This profile may range from short (C₄- to C₆-fatty acids), to medium and/or long (C₈- and higher) chain fatty acids. The mixture of short (C₄- to C₆-fatty acids), medium and/or long (C₈- and higher) chain fatty acids obtained is dependent, at least, on the lipolytic enzyme and on the lipolytic substrate. The mixture of short (C₄- to C₆-fatty acids), medium and/or long (C₈- and higher) chain fatty acids obtained is responsible for modulating the flavor of a given final product. For example, a mixture having a higher amount or concentration of short chain fatty acids, such as a higher amount or concentration of butyric acid (C₄-fatty acid), than an amount or concentration of medium and/or long chain fatty acids leads to the development of a preferred rancid cheese flavor, in particular during ripening, and has been used for flavor generation in dairy products, for example in various cheese types. In contrast, a mixture having a higher amount or concentration of medium and/or long chain fatty acids, specially a mixture having a higher amount or concentration of long chain fatty acids (C₁₆- and higher), than an amount or concentration of short chain fatty acids gives rise to off flavors.

Lipolytic enzymes or lipases can be of animal origin or non-animal origin. Lipases from animal origin (kid goat, calf or lamb) preferably cleave short chain fatty acids (C₄- to C₆-fatty acids) from milk fat and/or other fat rather than long chain fatty acids. These short chain fatty acids, in particular C₄-fatty acids, are responsible for the formation of the preferred rancid taste rather than the soapy taste produced by the medium to long chain fatty acids. To lesser extent they also release medium chain fatty acids, which add animal specific taste to the cheese. Nevertheless, lipases from animal origin cannot be used in vegetarian and/or kosher products, which is a highly demanded market. On the other hand, lipases from non-animal origin, such as from microbial origin, fully fulfill the vegetarian and/or kosher requirements, however microbial lipases are known for being non-specific lipases mainly releasing medium to long chain fatty acids (C₈- and higher) from milk fat and/or other fat, thereby giving cheese an extremely unpleasant soapy taste.

Therefore, there is a driver in the food industry, especially in the cheese industry, to look for a new source of a microbial wild-type, non-engineered, lipase presenting a higher specificity towards short chain fatty acids, such as C₄-fatty acids and/or C₆-fatty acids, than towards medium or long chain fatty acids, and/or to look for a new source of a microbial wild-type, non-engineered, lipase presenting a higher specificity towards short chain fatty acids, such as C₄-fatty acids and/or C₆-fatty acids, and additionally a specificity towards medium chain fatty acids, such as C₈- or C₁₀-fatty acids, than towards long chain fatty acids.

Several attempts to replace animal lipases are known in the prior art. These attempts include the use of microbial lipases, recombinant lipases and/or genetic engineered lipases. However, these attempts have failed to deliver a microbial wild-type lipase with a higher specificity towards short chain fatty acids, preferably towards C₄-fatty acids, and a desirable flavor development in food products such as in cheese.

Microbial lipases can also be obtained from Yarrowia, such as Yarrowia (Y.) lipolytica. Several documents disclose the preference of lipases from Yarrowia towards medium to long chain fatty acids. For example, Kamoun et al. 2015 describes a higher specificity for trioctanoin than for tributyrin at pH 7.5 for Lip8; Aloulou et al. 2007 discloses a higher specificity for trioctanoin than for tributyrin at pH 6 for Lip2; Sheng, J., et al. 2012 mentions the LipY has the highest activity on C₅-C₁₂ fatty acids. However, these examples show Pichia pastoris, genetically modified Y. lipolytica strains or Escherichia coli, respectively, as the host for the production of the lipases from Yarrowia.

Furthermore, the demand for the use of non-genetically modified organisms for production of food elements is growing significantly.

The foregoing illustrates the difficulty in obtaining a microbial wild-type lipase with higher specificity towards short chain fatty acids, specially towards C₄-fatty acids, and a lower specificity towards medium and/or long chain fatty acids, specially a lower specificity towards long chain fatty acids, leading to a reduction in soapiness and an increase in butyric flavors in a food product, such as a dairy product or cheese, and therefore able to replace the microbial lipases currently available, while simultaneously the microbial lipase is produced in a non-genetically modified organism.

### SUMMARY OF THE INVENTION

The present invention relates to Yarrowia, in particular Yarrowia lipolytica, for the production of a wild-type homologous lipolytic enzyme suitable to be used in the food industry, preferably in the dairy industry, more preferably in the cheese industry, wherein the enzyme has a higher specificity towards short chain fatty acids, in particular C₄-fatty acids, when compared to a known microbial lipolytic enzyme under the same conditions, in particular for the same dosage of enzyme and in the same fat matrix, meaning that the enzyme releases or produces or generates more short chain fatty acids than a commercial microbial enzyme(s) or a reference microbial enzyme(s) or a control microbial enzyme(s). Preferably, the Yarrowia strain is a non-genetically modified strain, more preferably the Yarrowia lipolytic strain is a non-genetically modified strain.

This is unexpected as, until now, the known wild-type microbial lipolytic enzymes were known to be specific towards medium or long chain fatty acids and not short chain fatty acids, preferably not specific towards C₄-fatty acids, while its production is carried out in heterologous hosts. Thus, a wild-type microbial lipolytic enzyme expressed in a non-genetically modified organism having higher specificity towards short chain fatty acids is surprising in view of the prior art.

The present invention relates to a method for preparing a food product comprising the following steps:
a) expressing a lipase or mixture of lipases from Yarrowia in a Yarrowia cell or Yarrowia strain;
b) using the lipase or mixture of lipases of step a) for preparing the food product;
wherein the lipase or mixture of lipases has a higher specificity towards the release of C₄-fatty acids from a dairy composition comprising milk fat and/or other fat if compared with any other fatty acids, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10;
preferably wherein the Yarrowia cell or Yarrowia strain is a non-genetically modified Yarrowia cell or Yarrowia strain;
preferably wherein the Yarrowia strain is a Yarrowia lipolytica strain;
preferably wherein the food product has a higher specificity towards the release of C₄-fatty acids from a dairy composition comprising milk fat and/or other fat if compared with other fatty acids or if compared with the fatty acids release by SEQ ID NO: 9 or 10.

The invention also concerns the use of a Yarrowia strain as a producer of a lipase or mixture of lipases in a method for preparing a food product, wherein the lipase or mixture of lipases has a higher specificity towards the release of C₄-fatty acids from a dairy composition comprising milk fat and/or other fat if compared with any other fatty acids, in particular if compared with the release of C₆-fatty acids, or if compared with the release of C₁₂-fatty acids or if compared with the release of C_{18:2}-fatty acids or if compared with the release of C_{18:0}-fatty acids.

Further, the invention relates to a Yarrowia lipolytica strain selected from a list consisting of: DSM 33988, DSM 33987, DSM 33985, DSM 33986; a mutant of DSM 33988, a mutant of DSM 33987, a mutant of DSM 33985, a mutant of DSM 33986; a variant of DSM 33988, a mutant of DSM 33987, a mutant of DSM 33985, a mutant of DSM 33986; wherein the strain(s) DSM 33988, DSM 33987, DSM 33985, DSM 33986 has(have) been deposited at DSMZ on 18 of August of 2021.

Finally, the invention relates to a method for expressing a lipase from Yarrowia in a Yarrowia cell or Yarrowia strain, wherein the method comprises the following step:
expressing the lipase or mixture of lipases from Yarrowia in a Yarrowia cell or Yarrowia strain in a growth medium without a carbon source selected from sugars such as glucose, olive oil, tributyrin, or mixtures thereof, preferably without glucose, more preferably without added glucose;
wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10,
preferably wherein the Yarrowia cell or Yarrowia strain is a non-genetically modified Yarrowia cell or Yarrowia strain;
preferably wherein the Yarrowia strain is a Yarrowia lipolytica strain.

### Definitions

In the context of the present invention the terms “short chain fatty acid”, “medium chain fatty acid”, “long chain fatty acid”, “FFA”, “lipase”, “lipolytic enzyme”, “microbial lipase”, “microbial lipolytic enzyme”, “lipase with higher specificity towards the release of short-chain fatty acids”, “lipase with higher specificity towards the release of C₄-fatty acids”, “lipase with higher specificity towards the release of C₆-fatty acids”, “lipase with higher specificity towards the release of medium and/or long-chain fatty acids”, “lipase activity”, “lipase dosage”, “wild type”, “synthetic sequence”, “isolated sequence”, “recombinant sequence”, “mature protein sequence”, “expression vector”, “sequence identity”, “food product”, “dairy product”, “cheese”, “cheese product”, “processed cheese”, “enzyme-modified cheese”, “cheese-like product”, “improvement factor (IF)”, “control microbial lipase”, “signal peptide”, “propeptide” have the meaning explained below.

The term “short chain fatty acid” means a fatty acid comprising 4-6 carbons. Examples of short chain fatty acids are: butyric acid (butanoic acid; C₄), valeric acid (pentanoic acid; C₅) or caproic acid (hexanoic acid; C₆).

The term “medium chain fatty acid” means a fatty acid comprising 8-12 carbons. Examples of medium chain fatty acids are: caprylic acid (octanoic acid; C₈), pelargonic acid (nonanoic acid; C₉), capric acid (decanoic acid; C₁₀), undecylic acid (undecanoic acid; C₁₁) or lauric acid (dodecanoic acid; C₁₂).

The term “long chain fatty acid” means a fatty acid comprising 14-18 or more carbons. Examples of long chain fatty acids are: myristic acid (tetradecanoic acid; C₁₄), pentadecanoic acid (C₁₅), palmitic acid (hexadecanoic acid; C₁₆), margaric acid (heptadecanoic acid; C₁₇), stearic acid (octadecanoic acid; Cis).

The term “FFA” stands for free fatty acid.

The term “lipase” and “lipolytic enzyme” are used interchangeably.

The term “microbial lipase” and “microbial lipolytic enzyme” are used interchangeably, and as used herein means a lipase or a lipolytic enzyme expressed by a naturally occurring microorganism found in nature. Furthermore, the microorganism may be a filamentous fungus and/or a non-filamentous fungus. Thus, the lipase or the microbial lipase or the microbial lipolytic enzyme may be a filamentous fungus lipase and/or non-filamentous fungus lipase.

The term “lipase with higher specificity towards the release of short-chain fatty acids”, from a dairy composition comprising milk fat and/or other fat, means that said lipase preferably hydrolyzes ester bonds of lipids of triglycerides generating more C₄-fatty acids and/or more C₆-fatty acids rather than generating medium and/or long chain fatty acids.

The term “lipase with higher specificity towards the release of C₄-fatty acids”, from a dairy composition comprising milk fat and/or other fat, means that said lipase preferably hydrolyzes ester bonds of lipids of triglycerides generating more C₄-fatty acids rather than generating other kinds of fatty acids, such as medium and/or long chain fatty acids. Preferably, the term “lipase with higher specificity towards the release of C₄-fatty acids”, from a dairy composition comprising milk fat and/or other fat, if compared with the release of C₁₀-fatty acids means that said lipase preferably hydrolyses ester bonds of lipids of triglycerides generating more C₄-fatty acids rather than generating C₁₀-fatty acids. More preferably, the term “lipase with higher specificity towards the release of C₄-fatty acids”, from a dairy composition comprising milk fat and/or other fat, if compared with the release of C₈-fatty acids or C₆-fatty acids or C₁₂-fatty acids or C_{18:2}-fatty acids or C₁₄-fatty acids or C_{18:0}-fatty acids or C_{18:1}-fatty acids or C₁₆-fatty acids means that said lipase preferably hydrolyses ester bonds of lipids of triglycerides generating more C₄-fatty acids rather than generating C₈-fatty acids or C₆-fatty acids or C₁₂-fatty acids or C_{18:2}-fatty acids or C₁₄-fatty acids or C_{18:0}-fatty acids or C_{18:1}-fatty acids or C₁₆-fatty acids, respectively. In particular, the molar fraction of C₄-fatty acids is higher than the molar fraction of medium and/or long chain fatty acids, for the same tested conditions, preferably the molar fraction of C₄-fatty acids is higher than the molar fraction of other fatty acids, such as C₆-fatty acids or C₈-fatty acids or C₁₀-fatty acids or C₁₂-fatty acids or C₁₄-fatty acids or C₁₆-fatty acids or C_{18:0}-fatty acids or C_{18:1}-fatty acids or C_{18:2}-fatty acids.

The term “lipase with higher specificity towards the release of C₄-fatty acids”, from a dairy composition comprising milk fat and/or other fat, also means that said lipase preferably hydrolyzes ester bonds of lipids of triglycerides generating more C₄-fatty acids than a control microbial lipase. In particular, the molar fraction of C₄-fatty acids of the tested lipase is higher than the molar fraction of C₄-fatty acids of the control microbial lipase.

The term “lipase with higher specificity towards the release of C₆-fatty acids”, from a dairy composition comprising milk fat and/or other fat, means that said lipase preferably hydrolyzes ester bonds of lipids of triglycerides generating more C₆-fatty acids rather than generating other kinds of fatty acids, such as medium and/or long chain fatty acids. Preferably, the term “lipase with higher specificity towards the release of C₆-fatty acids”, from a dairy composition comprising milk fat and/or other fat, if compared with the release of C₁₀-fatty acids means that said lipase preferably hydrolyses ester bonds of lipids of triglycerides generating more C₆-fatty acids rather than generating C₁₀-fatty acids. More preferably, the term “lipase with higher specificity towards the release of C₆-fatty acids”, from a dairy composition comprising milk fat and/or other fat, if compared with the release of C₈-fatty acids or C₁₂-fatty acids or C_{18:2}-fatty acids or C₁₄-fatty acids or C_{18:0}-fatty acids or C_{18:1}-fatty acids or C₁₆-fatty acids means that said lipase preferably hydrolyses ester bonds of lipids of triglycerides generating more C₆-fatty acids rather than generating C₈-fatty acids or C₁₂-fatty acids or C_{18:2}-fatty acids or C₁₄-fatty acids or C_{18:0}-fatty acids or C_{18:1}-fatty acids or C₁₆-fatty acids, respectively. In particular, the molar fraction of C₆-fatty acids is higher than the molar fraction of medium and/or long chain fatty acids, for the same tested conditions, preferably the molar fraction of C₆-fatty acids is higher than the molar fraction of other fatty acids, such as C₈-fatty acids or C₁₀-fatty acids or C₁₂-fatty acids or C₁₄-fatty acids or C₁₆-fatty acids or C_{18:0}-fatty acids or C_{18:1}-fatty acids or C_{18:2}-fatty acids.

The term “lipase with higher specificity towards the release of C₆-fatty acids”, from a dairy composition comprising milk fat and/or other fat, also means that said lipase preferably hydrolyzes ester bonds of lipids of triglycerides generating more C₆-fatty acids than a control microbial lipase. In particular, the molar fraction of C₆-fatty acids of the tested lipase is higher than the molar fraction of C₆-fatty acids of the control microbial lipase.

The term “lipase with higher specificity towards the release of medium and/or long-chain fatty acids”, from a dairy composition comprising milk fat and/or other fat, means that said lipase preferably hydrolyzes ester bonds of lipids of triglycerides generating C₈- to C₁₈-fatty acids rather than generating C₄-fatty acids and/or C₆-fatty acids. In particular, the molar fraction of C₈- to C₁₈-fatty acids of the tested lipase is higher than the molar fraction of C₈- to C₁₈-fatty of the control microbial lipase.

In the context of the present inventions the following terms are interchangeable “C₄-fatty acids” and “C_{4:0}-fatty acids”; “C₆-fatty acids” and “C_{6:0}-fatty acids”; “C₈-fatty acids” and “C_{8:0}-fatty acids”; “C₁₀-fatty acids” and “C_{10:0}-fatty acids”; “C₁₂-fatty acids” and “C_{12:0}-fatty acids”; “C₁₄-fatty acids” and “C_{14:0}-fatty acids”. The term “C₁₆-fatty acids” may include C_{16:0}-fatty acids and C_{16:1}-fatty acids”. The term “C₁₈-fatty acids” may include C_{18:0}-fatty, C_{18:1}-fatty acids and C_{18:3}-fatty.

The term “lipase activity” and “lipase dosage” are used interchangeably. The “lipase activity” may be determined, for dosing in cheese and cream, for example by the International Dairy Federation (IDF) method for the commercial microbial lipase or reference microbial lipase or control microbial lipase. The IDF method, mainly, corresponds to the International Standard ISO 13082:2011, 1^{st} edition of 2011-11-15, IDF 218:2011 (also labelled as ISO 130821218). However, in the present invention, the term “LFU/L of milk” is used. The term “LFU/L of milk” relates to the concentration or amount of enzyme or dosage of enzyme per liter of milk used to produce a dairy product, such as cheese. In the context of the present invention “LFU/L of milk” means lipase forestomach units per liter of milk. One LFU is defined as the amount of lipase activity that releases butyric acid at a rate of 1.25 μmol/min, under the tested conditions. Further, the lipase activity may also be determined as explained in the Examples below. Finally, there are other methods of determining lipase activity which has been disclosed in prior art documents (such as in EP 3 081 644, EP 2 254 996 or EP 1 776 455). However, what is relevant is that within the same example or within comparative examples, the lipase activity is determined in the same way for all lipases under analysis, including the control lipase.

The term “wild type” lipase means, in the context of the present invention, a lipase whose sequence has not been mutated, i.e. does not contain amino acid deletions, additions or substitutions, when compared with a mature protein sequence (after co- and/or post-translational cleavage events) endogenously produced. Wild type lipases of the present invention may comprise a non-endogenous signal- and/or propeptide selected for expression in Yarrowia. Furthermore, in the present invention, a lipase may also be synthetically obtained or genetically modified, using methods well known in the art, as to reproduce the wild-type lipase.

The term “homologous” means that the enzyme is produced in its native host organism, i.e., in the microbial organism it naturally occurs in and originated from.

The term “non-genetically modified organism” means an organism or microorganism whose genetic material has not been modified by man using genetic engineering or transgenic technology, thereby creating a combination of genes that do not occur in nature or through traditional crossbreeding methods, mating, or natural recombination.

The term “isolated sequence” or a variation thereof means a sequence which is at least substantially free from at least one other component with which the sequence is naturally associated in nature and as found in nature. In alternative, an “isolated sequence” can also mean a sequence removed from its native environment. For example, sequences which have been recombinantly produced in a host organism are considered isolated for the purpose of this invention as are native or recombinant sequences which have been substantially purified by any suitable technique as such, for example, a purification method. Purification methods are well known in the art. The term applies to an isolated microbial lipase or to an isolated DNA sequence.

The term “mature protein sequence” used herein means a sequence having lipolytic activity that is in its final form following translation and any co- and/or post-translational cleavage events or modifications, such as N-terminal processing, C-terminal truncation, glycosylation, phosphorylation, among other events or modifications.

The term “sequence identity” describes a quantitative measure of similarity between two amino acid sequences or two nucleotide sequences. For purposes of the present invention, the degree of sequence identity between two amino acid sequences is based on aligning both sequences with the blastp suite provided by the National center for Biotechnology Information (NCBI) on https://blast.ncbi.nlm.nih.gov applying standard parameter settings (Matrix: BLOSUM62, Gap Costs: Existence: 11 Extension:1, Conditional compositional score matrix adjustment) and subsequent quantification of identical amino acid pairs in identical positions over the aligned amino acid sequences.

The term “food product” refers to a kind of milk-based product intended to be used as food, including, but not limited to, cheese, milk, skimmed milk, acidified milk, butter milk, condensed milk, spread, margarine, yoghurt, ice cream, milk powder, butter, dulce de leche, among others.

The term “dairy product” is intended to include any food product made using milk or milk-based product, including, but not limited to, cheese, milk, skimmed milk, acidified milk, butter milk, condensed milk, spread, margarine, yoghurt, ice cream, milk powder, butter, dulce de leche, among others.

The term “cheese” or “cheese product” are used interchangeably. The term “cheese” is understood to encompass any cheese, including, but not limited to, hard cheeses such as Pecorino, Provolone, Parmesan, Grana Padano, Parmigiano Reggiano, Romano, Chester, Danbo, Manchego, Saint Paulin, Cheddar, Monterey, Colby, Edam, Gouda, Muenster, Swiss type, Gruyere, Emmental; curd-cheeses such as Feta cheese; pasta filata cheeses such as Mozzarella, and Queso fresco cheese; fresh cheese such as Ricotta, Cream cheese, Neufchatel or Cottage cheese; cream cheese, white mold cheese such as Brie and Camembert cheese, blue mold cheese such as Gorgonzola and Danish blue cheese; and processed cheese, enzyme-modified cheese (EMC) or cheese-like product.

The term “processed cheese” is preferably manufactured from cheese or cheese analogues by cooking and emulsifying the cheese, such as with emulsifying salts (e.g. phosphates and citrate). The process may further include the addition of spices/condiments.

The term “enzyme-modified cheese” or “EMC” is understood as cheese curd which has been treated with enzymes to produce a concentrated cheese flavor ingredient which may have approximately 15-30 times the flavor intensity of natural cheese. EMCs are available as pastes or dried to form powders and are used to give a cheese flavor note to products such as processed cheese or analogue cheese, cheese powders, soups, sauces, dips, crackers, salad dressings and in coatings for snack foods.

The term “cheese-like product” is understood as cheese-like products which contain fat, such as e.g. milk fat (e.g. cream or butter) or vegetable oil, as a part of the composition, and which further contain, as part of the composition, one or more non-milk constituents, such as e.g. a vegetable constituent (e.g. vegetable protein or vegetable oil). In the context of the present invention, a “cheese-like product” includes a non-dairy cheese, also known as vegan cheese, wherein the fat used to make the non-dairy cheese is vegetable oil or an emulsion such as water-in-oil emulsion, instead of milk fat.

The term “improvement factor (IF)” is defined as the ratio obtained when dividing the enzymatic specificity for the release of short chain fatty acids of a tested lipase by the enzymatic specificity for the release of short chain fatty acids of a control lipase, preferably a control microbial lipase or is defined as the ratio obtained when dividing the enzymatic specificity for the release of medium chain fatty acids of a tested lipase by the enzymatic specificity for the release of medium chain fatty acids of a control lipase, preferably a control microbial lipase. If the ratio is 1, then the tested lipase has an identical preference, or specificity, for short chain fatty acids as control sequence. If the ratio is lower than 1, then the tested lipase has less preference, or specificity, for short chain fatty acids than the control lipase, preferably a control microbial lipase. If the ratio is higher than 1, then the tested lipase has higher preference, or specificity, for short chain fatty acids than the control. The enzymatic specificity for short chain fatty acids is obtained by dividing the enzymatic activity for short chain fatty acids C₄ or C₆ by the enzymatic activity for long chain fatty acids C₁₆-C₁₈.

The improvement factor (IF) may also be determined by the ratio obtained when dividing the enzymatic specificity for the release of long chain fatty acids of a tested lipase by the enzymatic specificity for the release of long chain fatty acids of a control lipase, preferably a control microbial lipase. If the ratio is 1, then the tested lipase has an identical preference, or specificity, for longer chain fatty acids as the control lipase, preferably a control microbial lipase. If the ratio is lower than 1, then the tested lipase has less preference, or specificity, for long chain fatty acids than the control. If the ratio is higher than 1, then the tested lipase has higher preference, or specificity, for long chain fatty acids than the control. The enzymatic specificity for long chain fatty acids is obtained by dividing the enzymatic activity for long chain fatty acids C₁₆-C₁₈ by the enzymatic activity for short chain fatty acids C₄ or C₆.

The term “control lipase” or “control microbial lipase” or “control lipolytic enzyme” or “control microbial lipolytic enzyme” is a commercial microbial lipase such as Palatase®, Palatase® 20000 L, available from Novozymes A/S, and herein labelled as commercial microbial lipase or commercial microbial lipase A or control microbial lipase A or reference microbial lipase A.

Alternatively, other microbial lipases can be used, for example SpiceIT® MPlus, available from Chr. Hansen A/S and herein labelled as commercial microbial lipase or commercial microbial lipase B or control microbial lipase B or reference microbial lipase B.

Amino acids are herein indicated by their one letter code or by their three letter code, both of which are well known to a person skilled in the art.

### DETAILED DESCRIPTION

The purpose of this invention is to provide a Yarrowia strain, preferably a Yarrowia lipolytica strain, as a non-genetically modified microbial expression organism capable of expressing a microbial wild-type lipase with a higher specificity towards the release of C₄-fatty acids from a dairy composition comprising milk fat and/or other fat, if compared with the release of medium and/or long chain fatty acids, preferably if compared with the release of long-chain fatty acids, or a higher specificity towards the release of C₄-fatty acids from a dairy composition comprising milk fat and/or other fat if compared to the release of C₄-fatty acids of a commercial microbial lipase or reference lipase, without the need to generate any mutation (addition, deletion and/or substitution) in the lipase sequence.

The present invention relates a Yarrowia strain for the production of a wild-type, non-engineered, microbial lipolytic enzyme with a high specificity for short-chain fatty acids. Preferably, the invention relates to Yarrowia lipolytica for the production of a homologous or native microbial lipolytic enzyme with a high specificity for short-chain fatty acids. More preferably, the invention relates to a non-genetically modified strain of Yarrowia or Yarrowia lipolytica, for the production of a wild-type, non-engineered, microbial lipolytic enzyme with a high specificity for short-chain fatty acids. Therefore, the microbial lipase fulfills the vegetarian and/or kosher requirements and is produced in a non-genetically modified organism while simultaneously presenting a specificity and/or preference for the cleavage of short chain fatty acids, preferably presenting a higher specificity towards C₄-fatty acids rather other kinds of fatty acids or presenting a higher specificity towards C₄-fatty acids than a commercial microbial lipase.

### First Aspect of the Invention

In aspect of the invention relates to a method for preparing a food product comprising the following steps:
a) expressing a lipase or mixture of lipases from Yarrowia, preferably Yarrowia lipolytica, in a Yarrowia cell or Yarrowia strain;
b) using the lipase or mixture of lipases of step a) for preparing the food product;
wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10;
preferably wherein the Yarrowia cell or Yarrowia strain is a non-genetically modified Yarrowia cell or Yarrowia strain, more preferably wherein the Yarrowia lipolytica cell or Yarrowia lipolytica strain is a non-genetically modified Yarrowia lipolytica cell or Yarrowia lipolytica strain;
preferably wherein the Yarrowia strain or Yarrowia cell is a Yarrowia lipolytica strain or Yarrowia lipolytica cell, respectively;
preferably wherein the food product has a higher specificity towards the release of C₄-fatty acids from a dairy composition comprising milk fat and/or other fat if compared with other fatty acids or if compared with the fatty acids release by SEQ ID NO: 9 or 10.

In a preferred embodiment, step a) may be carried out in the absence of carbon sources selected from sugars, olive oil, tributyrin, or mixtures thereof; preferably in the absence of carbon sources selected from glucose, olive oil, tributyrin, or mixtures thereof; more preferably in the absence of added glucose to a growth medium used for expressing the lipase or mixture of lipases from Yarrowia in the Yarrowia cell or Yarrowia strain.

In a preferred embodiment, step a) may be carried out in the absence of glucose and tributyrin or in the absence of glucose and olive oil, preferably in the absence of added glucose and tributyrin or in the absence of added glucose and olive oil to a growth medium used for expressing the lipase or mixture of lipases from Yarrowia in the Yarrowia cell or Yarrowia strain.

In a preferred embodiment, the lipase or the mixture of lipases may comprise an amino acid sequence having at least 90% identity with SEQ ID NO: 1 or SEQ ID NO: 2 or SEQ ID NO: 3 or SEQ ID NO: 4. In an more preferred embodiment, the lipase or the mixture of lipases may comprise an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

In a preferred embodiment, the lipase or mixture of lipases may have a higher specificity towards the release of C₄-fatty acids from a dairy composition comprising milk fat and/or other fat if compared with the release of C₆-fatty acids or C₁₂-fatty acids or C_{18:0}-fatty acids or C_{18:2}-fatty acids, at a pH below 7, preferably 6.6-6.8, or at a pH below 6, preferably at a pH between 3.8-5.6, more preferably at a pH between 4.4-5.4, even more preferably at a pH between 4.6-5.2; and/or the lipase or mixture of lipases has a higher specificity towards the release of C₄-fatty acids from a dairy composition comprising milk fat and/or other fat than SEQ ID NO: 5 at a pH below 7, preferably 6.6-6.8, or at a pH below 6, preferably at a pH between 3.8-5.6, more preferably at a pH between 4.4-5.4, even more preferably at a pH between 4.6-5.2.

In a preferred embodiment, the lipase or mixture of lipases may have a higher specificity towards the release of C₄-fatty acids from a dairy composition comprising milk fat and/or other fat if compared with any other fatty acid, in particular if compared with the release of C₆-fatty acids or C₁₂-fatty acids or C_{18:0}-fatty acids or C_{18:2}-fatty acids, at a temperature below 20° C., preferably below 15° C., more preferably below 10° C., even more preferably between 5-8° C.; and/or the lipase or mixture of lipases has a higher specificity towards the release of C₄-fatty acids from a dairy composition comprising milk fat and/or other fat than SEQ ID NO: 5 at a temperature below 20° C., preferably below 15° C., more preferably below 10° C., even more preferably between 5-8° C.

In a preferred embodiment, the food product may be a dairy product selected from a cheese, or a processed cheese, or a cheese-like product, or an enzyme-modified cheese, or a butter, or a yogurt, or a cream, or a seasoning; preferably cheese.

In a preferred embodiment, the dairy product may be a cheese selected from a list consisting of: Feta cheese; Provolone cheese; Pecorino cheese; Parmesan cheese; Grana Padano cheese; Parmigiano Reggiano cheese; Romano cheese; Chester cheese; Danbo cheese; Manchego cheese; Saint Paulin cheese; Cheddar cheese; Monterey cheese; Colby cheese; Edam cheese; Gouda cheese; Muenster cheese; Swiss type cheese; Gruyere cheese; Emmental cheese; pasta filata cheeses, preferably Mozzarella; Queso fresco cheese; fresh cheese, preferably Ricotta, cream cheese, Neufchatel or Cottage cheese; cream cheese; white mold cheese, preferably Brie or Camembert cheese; blue mold cheese, preferably Gorgonzola or Danish blue cheese.

In another preferred embodiment, the food product may be a non-dairy product, preferably a non-dairy cheese or vegan cheese.

In a preferred embodiment, the Yarrowia cell or Yarrowia strain is a non-genetically modified Yarrowia cell or Yarrowia strain.

In a preferred embodiment, the Yarrowia cell or Yarrowia strain is a Yarrowia lipolytica cell or Yarrowia lipolytica strain.

In a preferred embodiment, the Yarrowia strain is a Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a mutant of DSM 33986 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

In a preferred embodiment, the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, may be obtained by a conventional breeding technique or may be genetically-engineered.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, may express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may express a lipase or mixture of lipases having a higher specificity towards the release of C₄-fatty acids from a dairy composition comprising milk fat and/or other fat than SEQ ID NO: 10 or any well-known prior art lipase.

In a preferred embodiment, the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 90% identity with SEQ ID NO: 1 or SEQ ID NO: 2 or SEQ ID NO: 3 or SEQ ID NO: 4. In an even more preferred embodiment, the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

### Second Aspect of the Invention

This invention also relates to the use of a Yarrowia strain, preferably Yarrowia lipolytica strain, as a producer of a lipase or mixture of lipases in a method for preparing a food product, wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

Furthermore, the invention also concerns the use of a Yarrowia strain, preferably Yarrowia lipolytica strain, as a producer of a lipase or mixture of lipases in a method for preparing a food product, wherein the lipase or mixture of lipases has a higher specificity towards the release of C₄-fatty acids from a dairy composition comprising milk fat and/or other fat than SEQ ID NO: 9 or 10 or any well-known prior art lipase.

In a preferred embodiment, the lipase or mixture of lipases may comprise an amino acid sequence having at least 90% identity with SEQ ID NO: 1 or SEQ ID NO: 2 or SEQ ID NO: 3 or SEQ ID NO: 4. In a more preferred embodiment, the lipase or the mixture of lipases may comprise an amino acid sequence having at least an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

In a preferred embodiment, the food product may be a dairy product selected from a cheese, or a processed cheese, or a cheese-like product, or an enzyme-modified cheese, or a butter, or a yogurt, or a cream, or a seasoning, preferably the dairy product may be cheese.

In a preferred embodiment, the dairy product is a cheese selected from a list consisting of: Feta cheese; Provolone cheese; Pecorino cheese; Parmesan cheese; Grana Padano cheese; Parmigiano Reggiano cheese; Romano cheese; Chester cheese; Danbo cheese; Manchego cheese; Saint Paulin cheese; Cheddar cheese; Monterey cheese; Colby cheese; Edam cheese; Gouda cheese; Muenster cheese; Swiss type cheese; Gruyere cheese; Emmental cheese; pasta filata cheeses, preferably Mozzarella; Queso fresco cheese; fresh cheese, preferably Ricotta, cream cheese, Neufchatel or Cottage cheese; cream cheese; white mold cheese, preferably Brie or Camembert cheese; blue mold cheese, preferably Gorgonzola or Danish blue cheese.

In another preferred embodiment, the food product may be a non-dairy product, preferably a non-dairy cheese or vegan cheese.

In a preferred embodiment, the Yarrowia cell or Yarrowia strain is a Yarrowia lipolytica cell or Yarrowia lipolytica strain.

In a preferred embodiment, the Yarrowia strain is a Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a mutant of DSM 33986 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

In a preferred embodiment, the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, may be obtained by a conventional breeding technique or may be genetically-engineered.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, may express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may express a lipase or mixture of lipases having a higher specificity towards the release of C₄-fatty acids from a dairy composition comprising milk fat and/or other fat than SEQ ID NO: 9 or 10 or any well-known prior art lipase.

### Third Aspect of the Invention

The invention also relates to a Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a mutant of DSM 33986 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

In a preferred embodiment, the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, may be obtained by a conventional breeding technique or may be genetically-engineered.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, may express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may express a lipase or mixture of lipases having a higher specificity towards the release of C₄-fatty acids from a dairy composition comprising milk fat and/or other fat than SEQ ID NO: 10 or any well-known prior art lipase.

In a preferred embodiment, the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 90% identity with SEQ ID NO: 1 or SEQ ID NO: 2 or SEQ ID NO: 3 or SEQ ID NO: 4. In an even more preferred embodiment, the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

### Fourth Aspect of the Invention

Another aspect of the invention concerns a method for expressing a lipase from Yarrowia in a Yarrowia cell or Yarrowia strain, wherein the method comprises the following step:
expressing the lipase or mixture of lipases from Yarrowia, preferably Yarrowia lipolytica, in a Yarrowia cell or Yarrowia strain in a growth medium without a carbon source selected from sugars, olive oil, tributyrin, or mixtures thereof, preferably without glucose, preferably without added glucose;
wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10,
preferably wherein the Yarrowia cell or Yarrowia strain is a non-genetically modified Yarrowia cell or Yarrowia strain, more preferably wherein the Yarrowia lipolytica cell or Yarrowia lipolytica strain is a non-genetically modified Yarrowia lipolytica cell or Yarrowia lipolytica strain.

In a preferred embodiment, the expression of the lipase or mixture of lipases from Yarrowia may be carried out in the absence of carbon sources selected from sugars, olive oil, tributyrin, or mixtures thereof; preferably in the absence of carbon sources selected from glucose, olive oil, tributyrin, or mixtures thereof; more preferably in the absence of added glucose to a growth medium used for expressing the lipase or mixture of lipases from Yarrowia in the Yarrowia cell or Yarrowia strain.

In a preferred embodiment, the expression of the lipase or mixture of lipases from Yarrowia may be carried out in the absence of glucose and tributyrin or in the absence of glucose and olive oil, preferably in the absence of added glucose and tributyrin or in the absence of added glucose and olive oil to a growth medium used for expressing the lipase or mixture of lipases from Yarrowia in the Yarrowia cell or Yarrowia strain.

In a preferred embodiment, the lipase may comprise an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4, or the mixture of lipases may comprise at least an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

In a preferred embodiment, the Yarrowia cell or Yarrowia strain is a Yarrowia lipolytica cell or Yarrowia lipolytica strain.

In a preferred embodiment, the Yarrowia strain is a Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a mutant of DSM 33986 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

In a preferred embodiment, the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, may be obtained by a conventional breeding technique or may be genetically-engineered.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, may express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

In a preferred embodiment, DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may express a lipase or mixture of lipases having a higher specificity towards the release of C₄-fatty acids from a dairy composition comprising milk fat and/or other fat than SEQ ID NO: 9, 10 or any well-known prior art lipase.

In a preferred embodiment, the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 90% identity with SEQ ID NO: 1 or SEQ ID NO: 2 or SEQ ID NO: 3 or SEQ ID NO: 4. In an even more preferred embodiment, the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may comprise an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

Mutant(s) and/or Variants(s)

An embodiment common to all aspects of the invention concerns the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, which may be obtained by a conventional breeding technique or may be genetically-engineered. The mutant(s) or variant(s) obtained by a conventional breeding technique or genetically-engineered is(are) functionally equivalent to the deposited strains in terms of the specificity towards the release of C₄-fatty acids from a dairy composition comprising milk fat and/or other fat or any other fat matrix. To determine if said mutant(s) or variant(s) is functionally equivalent to the deposited strains, the mutant(s) or variant(s) has to be tested for its capacity and specificity of releasing of more C₄-fatty acids from a dairy composition comprising milk fat and/or other fat than any other fatty acid, as well as their capacity to maintain said capability and specificity over time. The explanation of how to determine the “lipase activity” has been extensively described above; furthermore, the Examples herein disclosed also explain how the specificity towards the release of C₄-fatty acids from a dairy composition comprising milk fat and/or other fat or any other fat matrix can be determined.

### EXAMPLES

### Example 1—Fermentation Medium Composition

Yarrowia strains 1-4 are isolates from food spoilage. The strains were grown on different media as to determine the conditions leading to the highest lipase activity, in particular when tributyrin is used as a substrate for lipase activity indicating the release of more short chain fatty acids, in particular C₄-fatty acids, than medium and/or long fatty acids.

The media tested differed in terms of carbon sources. Different carbon sources, including 5 g/L glucose, 10 g/L olive oil, and 10 g/L tributyrin, were tested to maximize lipase expression yields in fermentations of strains 1 and 2.

Yarrowia strains 1 and 2 were independently grown as follows. Strains were streaked on YPD agar plates (Invitrogen) and incubated at 28° C. for three days. Seed cultures were made by inoculating 3 mL YPD medium with a single colony of the respective strain and incubation at 28° C. and 225 rpm shaking for 16 hours. Seed cultures were diluted to a OD₆₀₀ of 0.2 in main fermentation media containing 10 g/L yeast extract and 20 g/L tryptone (medium 1) or 10 g/L yeast extract and 20 g/L peptone (medium 2) or 10 g/L yeast extract, 20 g/L tryptone, 10 g/L tributyrin, 5 g/L glucose (medium 3) or 10 g/L yeast extract, 20 g/L tryptone, 10 g/L olive oil, 5 g/L glucose (medium 4). Main fermentation proceeded at 28° C. and 225 rpm shaking for 24 hours. Fermentation supernatants were separated from cells by centrifugation at 3000 rpm for 5 min and 0.22 μm filter sterilization. Filtered supernatants were used for determining lipase activity without further treatment.

Lipase activity was determined with tributyrin as substrate as follows. A tributyrin/agarose emulsion was generated by dissolving 1.4% low-melting agarose in 250 mM sodium acetate buffer pH 5.25 including 40 mM CaCl₂, addition of 1% (v/v) tributyrin to the solubilized agarose at 60° C., and subsequent emulsification for 2 min using an IKA T25 Ultra-Turrax emulsifier at speed 13.5. A total volume of 50 μL emulsion was transferred to each well of a transparent 96-well assay plate and allowed to solidify at room temperature. Lipase reaction was started by adding 20 μL enzyme on top of the emulsion layer, in particular wherein 20 μL enzyme correspond to 20 μL of filtrate obtained from Example 1. Clearance of turbidity of the emulsion by enzymatic hydrolysis of substrate tributyrin was followed by measuring absorbance at 600 nm in a plate reader. Each assay was calibrated with various dilutions of commercial microbial lipase A with known enzymatic activity.

TABLE 1
Lipase activity in filtrates of strains 1 and 2 in
various medium compositions:
medium 1: 10 g/L yeast extract, 20 g/L tryptone;
medium 2: 10 g/L yeast extract, 20 g/L peptone;
medium 3: 10 g/L yeast extract, 20 g/L tryptone,
10 g/L tributyrin, 5 g/L glucose;
medium 4: 10 g/L yeast extract, 20 g/L tryptone,
10 g/L olive oil, 5 g/L glucose.

| strain | medium 1 | medium 2 | medium 3 | medium 4 |
|---|---|---|---|---|
| 1 | 1207 ± 147 | 1353 ± 29 | 294 ± 42 | 442 ± 61 |
| 2 | 935 ± 91 | 1526 ± 149 | 422 ± 115 | 468 ± 46 |

Both strains 1 and 2 yielded highest lipase activity in fermentation media wherein no addition of carbon sources selected from glucose, tributyrin and/or olive oil was made. Similar results are expected for strains 3 and 4.

### Example 2—Expression

The fermentation of Yarrowia strains, herein disclosed as strains 1, 2, 3 and 4 was independently carried out as follows. Strains were streaked on YPD agar plates (Invitrogen) and incubated at 28° C. for three days. Seed cultures were made by inoculating 3 mL YPD medium with a single colony of the respective strain and incubation at 28° C. and 225 rpm shaking for 16 hours. Seed cultures were diluted to a OD₆₀₀ of 0.2 in main fermentation media containing 10 g/L yeast extract and 20 g/L tryptone (medium 1 of Example 1) or 10 g/L yeast extract and 20 g/L peptone (medium 2 of Example 1). Main fermentation proceeded at 28° C. and 225 rpm shaking for 24 hours. Fermentation supernatants were separated from cells by centrifugation at 3000 rpm for 5 min and 0.22 μm filter sterilization. Filtered supernatants were used for enzymatic activity determination in cream and cheese production directly without further treatment.

Microbial lipase C was used in this example as a control (control microbial lipase C or reference microbial lipase C) and produced as follows. A codon-optimized nucleotide sequence encoding for SEQ ID NO: 9 was integrated into a targeted locus of the expression host Pichia pastoris. The pro part was cleaved by a protease of Pichia pastoris upon secretion leading to SEQ ID NO: 10. Microbial lipase C corresponds to microbial lipase A expressed in a different organism.

The obtained recombinant Pichia pastoris strain was grown on YPD agar plates (Invitrogen) under selective conditions (100 μg/mL Zeocin) at 30° C. for 16 h. Seed fermentation was performed by inoculating 3 mL YPD liquid medium (Invitrogen) including 100 μg/mL Zeocin with a single colony from the respective YPD agar plates, followed by incubation at 30° C. and 250 rpm agitation for 16 h. Main fermentation was performed in 4 mL total volume by diluting seed fermentations with BMGY medium (Invitrogen) including 100 μg/mL Zeocin to an OD₆₀₀ of 0.2, followed by incubation at 30° C. and 225 rpm agitation for 3 days in 24-well culture plates. The main fermentation sample was centrifuged to precipitate expression host cells and the supernatant was sterile filtered at 0.22 μm. The filtrate was used directly for lipase enzymatic activity characterization without further treatment. Lipase activity in the filtrate was determined by method 218:2011 of the International Dairy Federation (International Standard ISO 13082:2011, 1^{st} edition of 2011-11-15, as explained above).

### Example 3—Enzymatic Activity in Cream

The filtrates obtained in Example 2 were analyzed for their enzymatic activity towards milk fat and the released free fatty acids were quantified (Tables 2 and 3). The same procedure was conducted for control microbial lipase B or control microbial lipase C.

Lipase activity on milk fat in cream was carried out as follows. Fresh whipping cream (380/fat) and equal amounts of water were mixed by stirring for 30 min. Enzymatic reaction was started by adding 1-5 mL of filtrates of strains 1 and 2 or 80 LFU/L (final concentration) of microbial lipases B or C to 50 mL cream/water mix and proceeded at 37° C. for 20 hours. Subsequently, free fatty acids were extracted from the reaction and quantified by gas chromatography-mass spectrometry (GC-MS), using standard protocols in the art for quantifying fatty acids. Alternatively, the disclosure of Jong C., de and Badings H. T. in J. High Resolution Chromatography. 13, 84-98 (1990) can also be used. Additionally, free fatty acid profile in cream can also be made at the French Institute for Fats and Oils (ITERG).

Obtained fatty acid quantities of lipase reactions were corrected for the respective quantities determined for a similar cream sample without added lipase.

TABLE 2
Molar fractions of fatty acids released in cream by reference microbial lipase C,
reference microbial lipase B, and filtrates of strains 1-3, and respective improvement factor
over the reference microbial lipase C (IF4C) and B (IF4B).

| | C_{4:0} | C_{6:0} | C_{8:0} | C_{10:0} | C_{12:0} | C_{14:0} | C_{16:0} | C_{18:0} | C_{18:1} | C_{18:2} | IF4B | IF4C |
|---|---|---|---|---|---|---|---|---|---|---|---|---|
| C | 1.3 | 1.3 | 2.5 | 2.7 | 4.2 | 10.7 | 39.9 | 10.1 | 25.9 | 1.4 | 0.4 | 1.0 |
| B | 3.3 ± | 2.5 ± | 5.7 ± | 2.0 ± | 4.7 ± | 11.5 ± | 42.0 ± | 8.2 ± | 18.5 ± | 1.6 ± | 1.0 | 2.7 |
| | 1.1 | 0.9 | 0.3 | 0.1 | 0.3 | 0.3 | 3.7 | 0.7 | 5.0 | 1.1 | | |
| 1 | 7.0 ± | 3.0 ± | 9.0 ± | 9.1 ± | 4.9 ± | 7.4 ± | 20.3 ± | 6.0 ± | 32.3 ± | 1.1 ± | 2.5 | 6.7 |
| | 0.8 | 0.0 | 0.2 | 0.9 | 0.4 | 0.6 | 1.7 | 0.3 | 3.1 | 0.2 | | |
| 2 | 15.6 ± | 4.4 ± | 9.2 ± | 9.8 ± | 5.5 ± | 6.7 ± | 15.7 ± | 4.5 ± | 27.6 ± | 1.0 ± | 6.7 | 18.3 |
| | 0.9 | 0.9 | 0.1 | 1.3 | 0.0 | 0.1 | 3.2 | 0.1 | 0.2 | 0.3 | | |
| 3 | 8.4 | 1.5 | 4.1 | 6.7 | 6.0 | 10.6 | 29.4 | 8.3 | 21.1 | 3.8 | 2.8 | 7.7 |

Filtrates 1-3 generate more short chain fatty acids, in particular more butyric acid (C_{4:0}) than the reference microbial lipases B and C (Table 2) and can, therefore, be successfully used in a process for producing a food product wherein the presence of short chain fatty acid butyric acid (C_{4:0}) is needed and desired, such as for reduce soapiness and increase of butyric flavors in a food product. Further, filtrates 1 and 3 have similar profiles of butyric acid (C_{4:0}) while the profile of the remaining free fatty acids is different between filtrates 1 and 3.

The molar fractions of fatty acids released in cream of filtrates 2 and 4 is presented in Table 3. This table show filtrates 2 and 4 have similar profiles of free fatty acids.

TABLE 3
Molar fractions of fatty acids released in cream by filtrates of strains 2 and 4.

| | C_{4:0} | C_{6:0} | C_{8:0} | C_{10:0} | C_{12:0} | C_{14:0} | C_{16:0} | C_{18:0} |
|---|---|---|---|---|---|---|---|---|
| 2 | 15.2 ± | 3.3 ± | 6.6 ± | 7.8 ± | 7.0 ± | 14.7 ± | 32.3 ± | 13.1 ± |
| | 2.1 | 0.0 | 0.1 | 0.1 | 0.1 | 0.1 | 1.6 | 0.2 |
| 4 | 14.8 ± | 2.5 ± | 5.6 ± | 7.0 ± | 6.5 ± | 14.4 ± | 35.8 ± | 13.4 ± |
| | 2.2 | 0.0 | 0.0 | 0.0 | 0.0 | 0.1 | 2.0 | 0.3 |

### Example 4—Sensorial Evaluation

Filtrates obtained from strains 1 and 2 were further tested in cheese, in particular in Feta cheese aged 2-months, to evaluate flavor formation (Table 4). The cheese making process of Feta cheese, is well known in the art and to the skilled person. Cheeses were made with various amounts of filtrates of strains 1 and 2: 1 mL, 10 mL, 35 mL, and 100 mL. For reference, cheeses were made with either no lipase added, or 1.2 LFU/L commercial microbial lipase B. The enzyme activity of commercial microbial lipase B was determined by method 218:2011 of the International Dairy federation (ISO 13082:2011).

For sensorial evaluation of cheese, cheeses made with 100 mL filtrates of strains 1 and 2 were chosen, as they revealed similar overall taste intensity like the cheese with commercial microbial lipase B. Sensorial evaluation was performed by rating the taste according to sensational parameters butyric and soapy on a scale from 1 (none) to 6 (intense). The Feta cheese was produced using identical conditions no lipase (reference) or filtrate of strain 1 or filtrate of strain 2 or a commercial lipase (B).

TABLE 4
Sensorial evaluation carried out with Feta cheese aged 2-months.

| | Feta cheese aged 2-months | |
|---|---|---|
| | Butyric | Soapy |
| Reference (no lipase) | 0.5 | 0.5 |
| 1 | 4.0 | 0.8 |
| 2 | 3.8 | 0.8 |
| B | 4.5 | 3.3 |

Sensorial evaluation revealed similar butyric sensation of cheeses made with Yarrowia lipolytica strains and a reference microbial lipase, in particular from Mucor javanicus, but significantly less perception of soapiness in case of the Yarrowia samples (Table 4). Similar results are expected for filtrates of strains 3 and 4.

In conclusion, the present invention discloses that Yarrowia lipolytica can be used to express wild-type lipases from Yarrowia lipolytica with a higher specificity for short chain fatty acids, specially C₄-fatty acid, than the known prior art microbial lipases, leading to a reduction in soapiness and an increase in butyric flavors of a food product such as cheese, preferably when the lipase or mixture of lipases is/are expressed in the absence of carbon sources selected from sugars such as glucose, olive oil, tributyrin, or mixtures thereof, more preferably in the absence of carbon sources selected from glucose, olive oil, tributyrin, or mixtures thereof; even preferably in the absence of added glucose to a growth medium used for expressing the lipase or mixture of lipases. Additionally, this invention is made without resourcing to any further microorganisms. Therefore, this invention provides microbial wild-type lipases which fulfill the vegetarian and/or kosher requirements, while simultaneously being expressed in a non-genetically modified organism and showing higher specificity for short chain fatty acids, specially C₄-fatty acid and lower specificity for medium to long chain fatty acids from milk fat and/or other fat, specially showing lower specificity for long chain fatty acids from milk fat and/or other fat, thereby avoiding an extremely unpleasant soapy taste in a food product, like cheese.

The use of the terms “a” and “an” and “the” and similar references in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DEPOSIT AND EXPERT SOLUTION

The applicant requests that a sample of the deposited microorganisms stated below may only be made available to an expert, subject to available provisions governed by Industrial Property Offices of States Party to the Budapest Treaty, until the date on which the patent is granted.

TABLE 5
Deposits made at a Depositary institution having acquired
the status of international depositary authority under the
Budapest Treaty on the International Recognition of the
Deposit of Microorganisms for the Purposes of Patent
Procedure: Leibniz Institute DSMZ-German Collection of
Microorganisms and Cell Cultures Inhoffenstr. 7B,
38124 Braunschweig, Germany.

| Strain | Accession No. | Deposit date |
|---|---|---|
| Strain 1—Yarrowia lipolytica | DSM 33988 | 18 Aug. 2021 |
| Strain 2—Yarrowia lipolytica | DSM 33987 | 18 Aug. 2021 |
| Strain 3—Yarrowia lipolytica | DSM 33985 | 18 Aug. 2021 |
| Strain 4—Yarrowia lipolytica | DSM 33986 | 18 Aug. 2021 |

SEQUENCE LISTING

>SEQ ID NO: 1
MVLSTVIGEWFSRVLFGTVAPSPLTATAPISQDFYDTALTFSHLSNVAYCINTPLESLKSDFSCGVACSHFPNMELV
EEFGGEFFETSITGFLSIDHVKKEKYVVYRGTYDIGDVYTDIQLSQSPFLVTPSALGSTANLCEGCTIHDGWNKAYN
ETMGIIGDKLADHVNSNPDYRLVVTGHSLGAAIAVLSATSLKVNGQDPYLYTYGQPRIGNANFANFVSKQWFGEGDG
LSMDSDRRYFRLTHWNDLFVGFPAFKDYVHSVGEIYIDYFTVQPPLNKVFSCAGPESMSCYRKDFNALARLDIVKNH
LAYFDWISLCTLNIGRRDLERGRKFEGTWLYGGLANGSTIF
>SEQ ID NO: 2
AVLQKRVYTSTETSHIDQESYNFFEKYARLANIGYCVGPGTKIFKPFNCGLQCAHFPNVELIEEFHDPRLIFDVSGY
LAVDHASKQIYLVIRGTHSLEDVITDIRIMQAPLTNFDLAANISSTATCDDCLVHNGFIQSYNNTYNQIGPKLDSVI
EQYPDYQIAVTGHSLGGAAALLFGINLKVNDHDPLVVTLGQPIVGNAGFANWVDKLFFGQENPDVSKVSKDRKLYRI
THRGDIVPQVPFWDGYQHCSGEVFIDWPLIHPPLSNVVMCQGQSNKQCSAGNTLLQQVNVIGNHLQYFVTEGVCGI
>SEQ ID NO: 3
QTAPITQETYDFVLKYGWLSNVAYCVRAPGPFALQSDFTCGNSCAHFPDVTLDYQFGGNFFSTSVTGFLAHDHTKKE
KYIVFRGTFSIADAITDIQTIQQPYMTSIPPLNTTDINSTNPSASINCPGCQVHDGFQKAYRETMVNVQDRLVDFLG
NNTDYKLIVTGHSLGAVTALFMGINLKNLGYDPTLINYGQPRLGNKAFADYVDALFFKQGDDGLTINPERRMYRVTH
WNDFFVGWPAGYSHTLGEVYISDPTGINAPIEDVYSCAGPENNQCHHGSFNLLERLNILKNHCGYLNWIFYCAINVD
KREMMIDPPRVGKRVEHWSGKFSDVESTEGLMYEAIYPM
>SEQ ID NO: 4
MVSFGARIKDFFSVLLFGAASTSSSTKTALVSQGFYDAALDFSHLSNIAYCVNAPITPLKSDFSCGQSCVHFPDIEL
VHIFGGDFFSTSITGYLALDHVKKEKYVVERGTFSIADAITDIQFQQSSFLVNVPALNTFTANDTAPEAQIDCKQCK
IHDGFSKAFTETWHNIGDLLEQHLDSYPDYQLYVTGHSLGAAMALLGATSIKLRGYDPILINYGQPRVGNKAFADYI
SALWFGNGDGLEINQQRRLYRMTHWNDVFVGLPNWDGYTHSNGEVYIKGKYVNPPLKDVFSCAGGENSKCYRSEFNL
LAQINLLQNHLCYIDYIGFCALNVGRRELNDLPHYNGPYKYGHKTEEQFIAEGLELSN
>SEQ ID NO: 5
ATGGTTTTGTCTACTGTTATTGGTGAATGGTTCTCTAGAGTTTTGTTTGGTACTGTTGCTCCATCTCCTTTGACTGC
TACTGCTCCAATCTCTCAAGATTTCTACGATACTGCTTTGACTTTTTCTCATTTGTCTAACGTTGCTTACTGTATCA
ACACTCCATTGGAATCTTTGAAGTCTGATTTCTCTTGTGGTGTTGCTTGTTCTCACTTTCCTAACATGGAGTTGGTT
GAAGAGTTCGGTGGTGAATTTTTCGAGACTTCTATTACTGGTTTCTTGTCTATCGATCATGTTAAGAAAGAGAAGTA
CGTTGTTTACAGAGGTACTTACGATATCGGAGATGTTTACACTGATATCCAATTGTCTCAATCTCCATTCTTGGTTA
CTCCTTCTGCTTTGGGTTCTACTGCTAATTTGTGTGAAGGTTGTACTATTCACGATGGTTGGAACAAGGCTTATAAT
GAGACTATGGGTATTATTGGAGATAAATTGGCTGATCATGTTAACTCTAATCCTGATTACAGATTGGTTGTTACTGG
TCACTCTTTGGGTGCTGCTATTGCTGTTTTGTCTGCTACTTCTTTGAAGGTTAACGGTCAAGATCCATACTTGTATA
CTTACGGTCAACCTAGAATCGGTAACGCTAACTTCGCTAACTTCGTTTCTAAGCAATGGTTTGGTGAAGGAGATGGT
TTGTCTATGGATTCTGATAGAAGATACTTCAGATTGACTCATTGGAACGATTTGTTTGTTGGTTTCCCAGCTTTTAA
GGATTACGTTCACTCTGTTGGTGAAATCTACATCGATTACTTCACTGTTCAACCACCTTTGAACAAGGTTTTCTCTT
GTGCTGGTCCTGAGTCTATGTCTTGTTACAGAAAGGATTTCAACGCTTTGGCTAGATTGGATATCGTTAAAAATCAT
TTGGCTTACTTCGATTGGATTTCTTTGTGTACTTTGAACATTGGTAGAAGAGATTTGGAAAGAGGTAGAAAGTTTGA
GGGTACTTGGTTGTATGGTGGTTTGGCTAATGGTTCTACTATTTTC
>SEQ ID NO: 6
GCTGTTTTGCAAAAGAGAGTTTACACTTCTACTGAAACTTCTCATATCGATCAAGAATCTTACAACTTTTTCGAGAA
GTATGCTAGATTGGCTAATATTGGTTATTGTGTTGGTCCAGGTACTAAGATTTTTAAGCCTTTCAACTGTGGTTTGC
AATGTGCTCATTTCCCAAACGTTGAATTGATCGAAGAGTTTCACGATCCTAGATTGATTTTCGATGTTTCTGGTTAC
TTGGCTGTTGATCATGCTTCTAAGCAAATCTACTTGGTTATTAGAGGTACTCACTCTTTGGAGGATGTTATCACTGA
TATCAGAATCATGCAAGCTCCATTGACTAACTTTGATTTGGCTGCTAATATTTCTTCTACTGCTACTTGTGATGATT
GTTTGGTTCACAACGGTTTCATTCAATCTTACAACAACACTTACAATCAAATTGGTCCAAAGTTGGATTCTGTTATT
GAGCAATACCCTGATTATCAAATTGCTGTTACTGGTCATTCTTTGGGTGGTGCTGCTGCTTTGTTGTTCGGTATTAA
CTTGAAGGTTAATGATCACGATCCATTGGTTGTTACTTTGGGTCAACCTATTGTTGGTAACGCTGGTTTCGCTAATT
GGGTTGATAAGTTGTTTTTCGGTCAAGAAAACCCAGATGTTTCTAAGGTTTCTAAGGATAGAAAGTTGTACAGAATC
ACTCATAGAGGAGATATTGTTCCACAAGTTCCTTTTTGGGATGGTTATCAACATTGTTCTGGAGAGGTTTTCATTGA
TTGGCCTTTGATTCACCCACCTTTGTCTAATGTTGTTATGTGTCAAGGTCAATCTAACAAACAATGTTCTGCTGGTA
ACACTTTGTTGCAACAAGTTAACGTTATTGGTAATCACTTGCAATACTTCGTTACTGAAGGTGTTTGTGGTATT
>SEQ ID NO: 7
CAAACTGCTCCAATCACTCAAGAAACTTACGATTTCGTTTTGAAGTACGGTTGGTTGTCTAACGTTGCTTACTGTGT
TAGAGCTCCAGGTCCTTTTGCTTTGCAATCTGATTTCACTTGTGGTAACTCTTGTGCTCATTTCCCTGATGTTACTT
TGGATTACCAATTCGGTGGTAACTTTTTCTCTACTTCTGTTACTGGTTTCTTGGCTCACGATCACACTAAGAAAGAA
AAGTACATCGTTTTTAGAGGTACTTTCTCTATCGCTGATGCTATTACTGATATCCAAACTATCCAACAACCATACAT
GACTTCTATTCCACCTTTGAACACTACTGATATCAACTCTACTAATCCATCTGCTTCTATTAATTGTCCTGGTTGTC
AAGTTCACGATGGTTTTCAAAAAGCTTACAGAGAGACTATGGTTAACGTTCAAGATAGATTGGTTGATTTCTTGGGT
AACAACACTGATTACAAGTTGATCGTTACTGGTCACTCTTTGGGTGCTGTTACTGCTTTGTTTATGGGTATTAACTT
GAAAAATTTGGGTTACGATCCAACTTTGATTAACTATGGTCAACCTAGATTGGGTAATAAGGCTTTCGCTGATTACG
TTGATGCTTTGTTTTTCAAACAAGGAGATGATGGTTTGACTATTAACCCAGAAAGAAGAATGTACAGAGTTACTCAT
TGGAACGATTTCTTTGTTGGTTGGCCTGCTGGTTACTCTCACACTTTGGGTGAAGTTTATATTTCTGACCCAACTGG
TATTAACGCTCCTATTGAAGATGTTTACTCTTGTGCTGGTCCAGAGAACAATCAATGTCATCACGGTTCTTTTAATT
TGTTGGAAAGATTGAACATCTTGAAGAATCATTGTGGTTACTTGAACTGGATTTTCTATTGTGCTATTAATGTTGAT
AAGAGAGAAATGATGATTGATCCACCTAGAGTTGGTAAAAGAGTTGAGCACTGGTCTGGTAAATTTTCTGATGTTGA
ATCTACTGAGGGTTTGATGTACGAGGCTATCTACCCTATG
>SEQ ID NO: 8
ATGGTCCTGTCCACTGTTATCGGAGAATGGTTTTCAAGAGTTTTGTTTGGTACTGTCGCCCCATCACCACTCACCGC
CACAGCCCCCATTTCTCAGGACTTCTACGATACCGCTCTCACGTTCTCTCACCTATCTAATGTTGCTTACTGTATCA
ACACCCCTCTTGAGTCCCTCAAGTCCGACTTTTCCTGCGGTGTCGCATGTTCTCACTTCCCAAACATGGAACTTGTT
GAAGAGTTCGGTGGAGAATTTTTCGAAACTTCTATTACTGGTTTTCTATCTATCGACCATGTCAAAAAGGAAAAGTA
CGTTGTGTACAGAGGCACCTATGACATTGGTGACGTTTATACTGACATTCAATTAAGCCAATCCCCATTCTTGGTTA
CCCCTTCTGCCCTGGGTTCTACAGCAAATCTGTGTGAGGGTTGTACTATCCACGATGGTTGGAACAAAGCATACAAC
GAAACCATGGGTATTATCGGAGACAAGCTCGCTGACCACGTTAACTCCAACCCAGATTACAGATTGGTCGTAACTGG
ACATTCACTTGGTGCTGCTATTGCTGTACTATCTGCAACCTCTCTTAAGGTCAATGGCCAAGATCCTTACCTTTACA
CTTACGGTCAGCCTCGTATTGGTAATGCTAACTTCGCAAACTTTGTGAGTAAGCAATGGTTCGGTGAGGGTGACGGT
CTATCTATGGACTCGGACAGACGTTATTTCAGACTGACTCACTGGAACGACCTCTTTGTTGGTTTCCCTGCTTTCAA
GGACTACGTGCACTCTGTCGGAGAGATTTACATAGACTATTTCACCGTTCAACCACCACTCAACAAAGTTTTCTCTT
GTGCTGGGCCTGAGTCTATGTCCTGTTACAGAAAGGACTTCAACGCCCTTGCTAGACTTGATATTGTGAAAAACCAC
CTAGCTTACTTCGATTGGATCTCTCTGTGCACCCTAAACATCGGCAGACGTGATCTGGAGAGGGGTAGAAAGTTCGA
GGGTACTTGGCTCTACGGTGGACTGGCTAACGGATCCACTATCTTC
>SEQ ID NO: 9
VPIKRQSNSTVDSLPPLIPSRTSAPSSSPSTTDPEAPAMSRNGPLPSDVETKYGMALNATSYPDSVVQAMSIDGGIR
AATSQEINELTYYTTLSANSYCRTVIPGATWDCIHCDATEDLKIIKTWSTLIYDTNAMVARGDSEKTIYIVFRGSSS
IRNWIADLTFVPVSYPPVSGTKVHKGFLDSYGEVQNELVATVLDQFKQYPSYKVAVTGHSLGGATALLCALGLYQRE
EGLSSSNLFLYTQGQPRVGDPAFANYVVSTGIPYRRTVNERDIVPHLPPAAFGFLHAGEEYWITDNSPETVQVCTSD
LETSDCSNSIVPFTSVLDHLSYFGINTGLCT
>SEQ ID NO: 10
SIDGGIRAATSQEINELTYYTTLSANSYCRTVIPGATWDCIHCDATEDLKIIKTWSTLIYDTNAMVARGDSEKTIYI
VFRGSSSIRNWIADLTFVPVSYPPVSGTKVHKGFLDSYGEVQNELVATVLDQFKQYPSYKVAVTGHSLGGATALLCA
LGLYQREEGLSSSNLFLYTQGQPRVGDPAFANYVVSTGIPYRRTVNERDIVPHLPPAAFGFLHAGEEYWITDNSPET
VQVCTSDLETSDCSNSIVPFTSVLDHLSYFGINTGLCT

### ITEMS

M1. Method for preparing a food product comprising the following steps:
a) expressing a lipase or mixture of lipases from Yarrowia, preferably Yarrowia lipolytica, in a Yarrowia cell or Yarrowia strain;
b) using the lipase or mixture of lipases of step a) for preparing the food product;
wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10.

M2. Method according to M1, wherein the food product has a higher specificity towards the release of C₄-fatty acids from a dairy composition comprising milk fat and/or other fat if compared with other fatty acids or if compared with the fatty acids release by SEQ ID NO: 9 or 10.

M3. Method according to any of M1-M2, wherein the Yarrowia cell or Yarrowia strain is a Yarrowia lipolytica cell or Yarrowia lipolytica strain.

M4. Method according to any of M1-M3, wherein the Yarrowia cell or Yarrowia strain is a non-genetically modified Yarrowia cell or Yarrowia strain.

M5. Method according to any of M1-M4, wherein the Yarrowia lipolytica cell or Yarrowia lipolytica strain is a non-genetically modified Yarrowia lipolytica cell or Yarrowia lipolytica strain.

M6. Method according to any of M1-M5, wherein step a) is carried out in the absence of carbon sources selected from sugars, olive oil, tributyrin, or mixtures thereof; preferably in the absence of carbon sources selected from glucose, olive oil, tributyrin, or mixtures thereof; more preferably in the absence of carbon sources selected from glucose and olive oil or selected from glucose and tributyrin; even more preferably in the absence of added glucose to a growth medium used for expressing the lipase or mixture of lipases from Yarrowia in the Yarrowia cell or Yarrowia strain.

M7. Method according to any of M1-M6, wherein step a) is carried out in the presence of yeast extract and tryptone and in the absence of glucose or olive oil or tributyrin.

M8. Method according to any of M1-M6, wherein step a) is carried out in the presence of yeast extract and peptone and in the absence of glucose or olive oil or tributyrin.

M9. Method according to any of M1-M8, wherein step a) is be carried out in the absence of glucose and tributyrin or in the absence of glucose and olive oil, preferably in the absence of added glucose and tributyrin or in the absence of added glucose and olive oil to a growth medium used for expressing the lipase or mixture of lipases from Yarrowia in the Yarrowia cell or Yarrowia strain.

M10. Method according to any of M1-M9, wherein the lipase or the mixture of lipases comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

M11. Method according to any of M1-M10, wherein the lipase or mixture of lipases has a higher specificity towards the release of C₄-fatty acids from a dairy composition comprising milk fat and/or other fat if compared with the release of C₆-fatty acids or C₁₂-fatty acids or C_{18:0}-fatty acids or C_{18:2}-fatty acids, at a pH below 7, preferably 6.6-6.8, or at a pH below 6, preferably at a pH between 3.8-5.6, more preferably at a pH between 4.4-5.4, even more preferably at a pH between 4.6-5.2; and/or the lipase or mixture of lipases has a higher specificity towards the release of C₄-fatty acids from a dairy composition comprising milk fat and/or other fat than SEQ ID NO: 5 at a pH below 7, preferably 6.6-6.8, or at a pH below 6, preferably at a pH between 3.8-5.6, more preferably at a pH between 4.4-5.4, even more preferably at a pH between 4.6-5.2.

M12. Method according to any of M1-M11, wherein the lipase or mixture of lipases has a higher specificity towards the release of C₄-fatty acids from a dairy composition comprising milk fat and/or other fat if compared with any other fatty acid, in particular if compared with the release of C₆-fatty acids or C₁₂-fatty acids or C_{18:0}-fatty acids or C_{18:2}-fatty acids, at a temperature below 20° C., preferably below 15° C., more preferably below 10° C., even more preferably between 5-8° C.; and/or the lipase or mixture of lipases has a higher specificity towards the release of C₄-fatty acids from a dairy composition comprising milk fat and/or other fat than SEQ ID NO: 5 at a temperature below 20° C., preferably below 15° C., more preferably below 10° C., even more preferably between 5-8° C.

M13. Method according to any of M1-M12, wherein the food product is a dairy product selected from a cheese, or a processed cheese, or a cheese-like product, or an enzyme-modified cheese, or a butter, or a yogurt, or a cream, or a seasoning; preferably cheese.

M14. Method according to M13, wherein the dairy product is be a cheese selected from a list consisting of: Feta cheese; Provolone cheese; Pecorino cheese; Parmesan cheese; Grana Padano cheese; Parmigiano Reggiano cheese; Romano cheese; Chester cheese; Danbo cheese; Manchego cheese; Saint Paulin cheese; Cheddar cheese; Monterey cheese; Colby cheese; Edam cheese; Gouda cheese; Muenster cheese; Swiss type cheese; Gruyere cheese; Emmental cheese; pasta filata cheeses, preferably Mozzarella; Queso fresco cheese; fresh cheese, preferably Ricotta, cream cheese, Neufchatel or Cottage cheese; cream cheese; white mold cheese, preferably Brie or Camembert cheese; blue mold cheese, preferably Gorgonzola or Danish blue cheese.

M15. Method according to any of M1-M12, wherein the food product is a non-dairy product, preferably a non-dairy cheese or vegan cheese.

M16. Method according to any of M1-M15, wherein the Yarrowia strain is a Yarrowia lipolytica strain selected from a list consisting of:
DSM 33988, DSM 33987, DSM 33985, DSM 33986,
a mutant of DSM 33988, a mutant of DSM 33987, a mutant of DSM 33985, a mutant of DSM 33986,
a variant of DSM 33988, a variant of DSM 33987, a variant of DSM 33985, a variant of DSM 33986,
wherein DSM 33988, DSM 33987, DSM 33985, DSM 33986 where deposited at DSMZ on 18 of August of 2021.

M17. Method according to M16, the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, is obtained by a conventional breeding technique or is be genetically-engineered.

M18. Method according to any of M16-M17, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, expresses a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

M19. Method according to any of M16-M18, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 expresses a lipase or mixture of lipases having a higher specificity towards the release of C₄-fatty acids from a dairy composition comprising milk fat and/or other fat than SEQ ID NO: 10 or any well-known prior art lipase.

M20. Method according to any of M16-M19, wherein the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprises an amino acid sequence having at least 90% identity with SEQ ID NO: 1 or SEQ ID NO: 2 or SEQ ID NO: 3 or SEQ ID NO: 4.

M21. Method according to any of M16-M20, wherein the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprises an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

M22. Method according to any of M1-21, wherein the lipase or mixture of lipases is encoding by a sequence comprising at least 90%, or at least 95%, or at least 96%, or at least 97%, or at least 98%, or at least 99% or 100% identity with SEQ ID NO: 5 or SEQ ID NO: 6 or SEQ ID NO: 7 or SEQ ID NO: 8.

U1. Use of a Yarrowia strain, preferably Yarrowia lipolytica strain, as a producer of a lipase or mixture of lipases in a method for preparing a food product according to any of M1 to M21, wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or wherein the lipase or mixture of lipases has a higher specificity towards the release of C₄-fatty acids from a dairy composition comprising milk fat and/or other fat than SEQ ID NO: 9 or 10 or any well-known prior art lipase.

U2. Use according to U1, wherein the lipase or mixture of lipases comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

U3. Use according to any of U1-U2, wherein the food product is a dairy product selected from a cheese, or a processed cheese, or a cheese-like product, or an enzyme-modified cheese, or a butter, or a yogurt, or a cream, or a seasoning, preferably the dairy product is cheese.

U4. Use according to any of U1-U3, wherein the dairy product is a cheese selected from a list consisting of: Feta cheese; Provolone cheese; Pecorino cheese; Parmesan cheese; Grana Padano cheese; Parmigiano Reggiano cheese; Romano cheese; Chester cheese; Danbo cheese; Manchego cheese; Saint Paulin cheese; Cheddar cheese; Monterey cheese; Colby cheese; Edam cheese; Gouda cheese; Muenster cheese; Swiss type cheese; Gruyere cheese; Emmental cheese; pasta filata cheeses, preferably Mozzarella; Queso fresco cheese; fresh cheese, preferably Ricotta, cream cheese, Neufchatel or Cottage cheese; cream cheese; white mold cheese, preferably Brie or Camembert cheese; blue mold cheese, preferably Gorgonzola or Danish blue cheese.

U5. Use according to any of U1-U3, wherein the food product is a non-dairy product, preferably a non-dairy cheese or vegan cheese.

U6. Use according to any of U1-U5, wherein the Yarrowia cell or Yarrowia strain is a Yarrowia lipolytica cell or Yarrowia lipolytica strain.

U7. Use according to any of U1-U6, wherein the Yarrowia cell or Yarrowia strain is a non-genetically modified Yarrowia cell or Yarrowia strain.

U8. Use according to any of U1-U7, wherein the Yarrowia lipolytica cell or Yarrowia lipolytica strain is a non-genetically modified Yarrowia lipolytica cell or Yarrowia lipolytica strain.

U9. Use according to any of U1-U8, wherein the Yarrowia strain is a Yarrowia lipolytica strain selected from a list consisting of:
DSM 33988, DSM 33987, DSM 33985, DSM 33986,
a mutant of DSM 33988, a mutant of DSM 33987, a mutant of DSM 33985, a mutant of DSM 33986,
a variant of DSM 33988, a variant of DSM 33987, a variant of DSM 33985, a variant of DSM 33986,
wherein DSM 33988, DSM 33987, DSM 33985, DSM 33986 where deposited at DSMZ on 18 of August of 2021.

U10. Use according to any of U1-U9, wherein the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, is obtained by a conventional breeding technique or is be genetically-engineered.

U11. Use according to any of U1-U10, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, expresses a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

U12. Use according to any of U1-U11, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 expresses a lipase or mixture of lipases having a higher specificity towards the release of C₄-fatty acids from a dairy composition comprising milk fat and/or other fat than SEQ ID NO: 9 or 10 or any well-known prior art lipase.

U13. Use according to any of U1-U12, wherein the lipase or mixture of lipases is encoding by a sequence comprising at least 90%, or at least 95%, or at least 96%, or at least 97%, or at least 98%, or at least 99% or 100% identity with SEQ ID NO: 5 or SEQ ID NO: 6 or SEQ ID NO:7 or SEQ ID NO:8.

S1. Yarrowia lipolytica strain selected from a list consisting of: DSM 33988, DSM 33987, DSM 33985, DSM 33986, a mutant of DSM 33988, a mutant of DSM 33987, a mutant of DSM 33985, a mutant of DSM 33986, a variant of DSM 33988, a variant of DSM 33987, a variant of DSM 33985, a variant of DSM 33986, wherein DSM 33988, DSM 33987, DSM 33985, DSM 33986 where deposited at DSMZ on 18 of August of 2021.

S2. Strain according to 51, wherein the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, is obtained by a conventional breeding technique or is genetically-engineered.

S3. Strain according to any of S1-S2, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, may express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

S4. Strain according to any of S1-S3, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 may express a lipase or mixture of lipases having a higher specificity towards the release of C₄-fatty acids from a dairy composition comprising milk fat and/or other fat than SEQ ID NO: 10 or any well-known prior art lipase.

S5. Strain according to any of S1-S4, wherein the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprises an amino acid sequence having at least 90% identity with SEQ ID NO: 1 or SEQ ID NO: 2 or SEQ ID NO: 3 or SEQ ID NO: 4.

S6. Strain according to any of S1-S5, wherein the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprises an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or encodes a sequence comprising at least 90%, or at least 95%, or at least 96%, or at least 97%, or at least 98%, or at least 99% or 100% identity with SEQ ID NO: 5 or SEQ ID NO: 6 or SEQ ID NO: 7 or SEQ ID NO: 8.

Me1. Method for expressing a lipase from Yarrowia in a Yarrowia cell or Yarrowia strain, wherein the method comprises the following step:
expressing the lipase or mixture of lipases from Yarrowia, preferably Yarrowia lipolytica, in a Yarrowia cell or Yarrowia strain in a growth medium without a carbon source selected from sugars such as glucose, olive oil, tributyrin, or mixtures thereof, preferably without glucose, preferably without added glucose;
wherein the lipase or mixture of lipases has a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10,
preferably wherein the Yarrowia cell or Yarrowia strain is a non-genetically modified Yarrowia cell or Yarrowia strain, more preferably wherein the Yarrowia lipolytica cell or Yarrowia lipolytica strain is a non-genetically modified Yarrowia lipolytica cell or Yarrowia lipolytica strain.

Me2. Method according to Me1, wherein the expression of the lipase or mixture of lipases from Yarrowia is be carried out in the absence of carbon sources selected from sugars, olive oil, tributyrin, or mixtures thereof; preferably in the absence of carbon sources selected from glucose, olive oil, tributyrin, or mixtures thereof; more preferably in the absence of added glucose to a growth medium used for expressing the lipase or mixture of lipases from Yarrowia in the Yarrowia cell or Yarrowia strain.

Me3. Method according to any of Me1-Me2, wherein the expression of the lipase or mixture of lipases from Yarrowia is carried out in the absence of glucose and tributyrin or in the absence of glucose and olive oil, preferably in the absence of added glucose and tributyrin or in the absence of added glucose and olive oil to a growth medium used for expressing the lipase or mixture of lipases from Yarrowia in the Yarrowia cell or Yarrowia strain.

Me4. Method according to any of Me1-Me3, wherein the lipase may comprise an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4, or the mixture of lipases may comprise at least an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

Me5. Method according to any of Me1-Me4, wherein the Yarrowia cell or Yarrowia strain is a Yarrowia lipolytica cell or Yarrowia lipolytica strain.

Me6. Method according to any of Me1-Me5, wherein the Yarrowia strain is a Yarrowia lipolytica strain selected from a list consisting of:
DSM 33988, DSM 33987, DSM 33985, DSM 33986,
a mutant of DSM 33988, a mutant of DSM 33987, a mutant of DSM 33985, a mutant of DSM 33986,
a variant of DSM 33988, a variant of DSM 33987, a variant of DSM 33985, a variant of DSM 33986,
wherein DSM 33988, DSM 33987, DSM 33985, DSM 33986 where deposited at DSMZ on 18 of August of 2021.

Me7. Method according to any of Me1-Me6, wherein the mutant of DSM 33988 or the mutant of DSM 33987 or the mutant of DSM 33985 or the mutant of DSM 33986, or the variant of DSM 33988 or the variant of DSM 33987 or the variant of DSM 33985 or the variant of DSM 33986, is obtained by a conventional breeding technique or by genetically-engineered.

Me8. Method according to any of Me1-Me7, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986, express a lipase or mixture of lipases having a higher specificity towards the release of C₄-fatty acids from a dairy composition comprising milk fat and/or other fat if compared with any other fatty acid, in particular if compared with the release of C₆-fatty acids, or if compared with the release of C₁₂-fatty acids or if compared with the release of C_{18:2}-fatty acids or if compared with the release of C_{18:0}-fatty acids.

Me9. Method according to any of Me1-Me8, wherein DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 express a lipase or mixture of lipases having a higher specificity towards the release of C₄-fatty acids from a dairy composition comprising milk fat and/or other fat than SEQ ID NO: 9, 10 or any well-known prior art lipase.

Me10. Method according to any of Me1-Me9, wherein the lipase or mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprises an amino acid sequence having at least 90% identity with SEQ ID NO: 1 or SEQ ID NO: 2 or SEQ ID NO: 3 or SEQ ID NO: 4.

Me11. Method according to any of Me1-Me10, wherein the lipase or the mixture of lipases expressed by DSM 33988 or DSM 33987 or DSM 33985 or DSM 33986, or the mutant of DSM 33988 or the mutant of DSM 33987 or the mutant of DSM 33985 or the mutant of DSM 33986, or variant of DSM 33988 or variant of DSM 33987 or variant of DSM 33985 or variant of DSM 33986 comprise an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4 or encodes a sequence comprising at least 90%, or at least 95%, or at least 96%, or at least 97%, or at least 98%, or at least 99% or 100% identity with SEQ ID NO: 5 or SEQ ID NO: 6 or SEQ ID NO: 7 or SEQ ID NO: 8.

### REFERENCES

### Non Patent Literature

Kamoun, J. et al. Biochemical characterization of Yarrowia lipolytica LIP8, a secreted lipase with a cleavable C-terminal region. Biochim. Biophys. Acta 1851, 129-140 (2015) Aloulou, A. et al. Purification and biochemical characterization of the LIP2 lipase from Yarrowia lipolytica. Biochim. Biophys. Acta—Mol. Cell Biol. Lipids 1771, 228-237 (2007) Sheng, J., Wang, F., Wang, H. & Sun, M. Cloning, characterization and expression of a novel lipase gene from marine psychotropic Yarrowia lipolytica. Ann. Microbiol. 62, 1071-1077 (2012) Jong C., de and Badings H. T. Determination of free fatty acids in milk and cheese procedures for extraction, clean up, and capillary gas chromatographic analysis J. High Resolution Chromatography, 13, 84-98 (1990)

### PATENT LITERATURE

EP3081644; EP2254996; EP1776455

1. A method for preparing a food product comprising the following steps:
a) expressing a lipase or mixture of lipases from Yarrowia, preferably Yarrowia lipolytica, in a Yarrowia cell or Yarrowia strain;
b) using the lipase or mixture of lipases of step a) for preparing the food product;
wherein step a) is carried out in the absence of carbon sources selected from sugars, olive oil, tributyrin, or mixtures thereof;
wherein the lipase comprises an amino acid sequence having at least 90% identity with SEQ ID NO: 1 or SEQ ID NO: 2 or SEQ ID NO: 3 or SEQ ID NO: 4, or wherein the mixture of lipases comprises at least an amino acid sequence having at least 90% identity with SEQ ID NO: 1 or SEQ ID NO: 2 or SEQ ID NO: 3 or SEQ ID NO: 4;
wherein the lipase or mixture of lipases has a higher specificity towards the release of C₄-fatty acids from a dairy composition comprising milk fat and/or other fat if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10;
preferably wherein the Yarrowia cell or Yarrowia strain is a Yarrowia lipolytica cell or a Yarrowia lipolytica strain.

2. The method according to claim 1, wherein step a) is carried out in the absence of carbon sources selected from glucose, olive oil, tributyrin, or mixtures thereof; preferably in the absence of carbon sources selected from glucose and olive oil or selected from glucose and tributyrin; more preferably in the absence of added glucose to a growth medium used for expressing the lipase or mixture of lipases from Yarrowia in the Yarrowia cell or Yarrowia strain.

3. The method according to claim 1, wherein the lipase comprises an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4, or wherein the mixture of lipases comprises at least an amino acid sequence having at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.

4. The method according to claim 1, wherein the food product is a dairy product selected from a cheese, or a processed cheese, or a cheese-like product, or an enzyme-modified cheese, or a butter, or a yogurt, or a cream, or a seasoning, preferably wherein the dairy product is a cheese selected from a list consisting of: Feta cheese; Provolone cheese; Pecorino cheese; Parmesan cheese; Grana Padano cheese; Parmigiana Reggiano cheese; Romano cheese; Chester cheese; Danbo cheese; Manchego cheese; Saint Paulin cheese; Cheddar cheese; Monterey cheese; Colby cheese; Edam cheese; Gouda cheese; Muenster cheese; Swiss type cheese; Gruyere cheese; Emmental cheese; pasta filata cheeses, preferably Mozzarella; Queso fresco cheese; fresh cheese, preferably Ricotta, cream cheese, Neufchatel or Cottage cheese; cream cheese; white mold cheese, preferably Brie or Camembert cheese; blue mold cheese, preferably Gorgonzola or Danish blue cheese.

5. The method according to claim 1, wherein the food product is a non-dairy product, preferably a non-dairy cheese or vegan cheese.

6. The method according to claim 1, wherein the Yarrowia cell or Yarrowia strain is a non-genetically modified Yarrowia lipolytica cell or Yarrowia lipolytica strain.

7. The method according to claim 1, wherein the Yarrowia strain is a Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a mutant of DSM 33986 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

8. The method according to claim 7, wherein the mutant of DSM 33988, DSM 33987, DSM 33985 or DSM 33986 or variant of DSM 33988, DSM 33987, DSM 33985 or DSM 33986 is obtained by a breeding technique.

9. The method according to claim 7, wherein DSM 33988 or DSM 33987 or DSM 33985 or DSM 33986, or the mutant of DSM 33988, DSM 33987, DSM 33985 or DSM 33986, or variant of DSM 33988, DSM 33987, DSM 33985 or DSM 33986 expresses a lipase or mixture of lipases having a higher specificity towards the release of C₄-fatty acids from a dairy composition comprising milk fat and/or other fat if compared with the release of C₆-fatty acids, or if compared with the release of C₁₂-fatty acids or if compared with the release of C_{18:2}-fatty acids or if compared with the release of C_{18:0}-fatty acids; and/or
wherein DSM 33988 or DSM 33987 or DSM 33985 or DSM 33986, or the mutant of DSM 33988, DSM 33987, DSM 33985 or DSM 33986, or variant of DSM 33988, DSM 33987, DSM 33985 or DSM 33986 expresses a lipase or mixture of lipases having a higher specificity towards the release of C₄-fatty acids from a dairy composition comprising milk fat and/or other fat than SEQ ID NO: 9 or 10.

10-13. (canceled)

14. A Yarrowia lipolytica strain selected from a list consisting of:
DSM 33988 deposited at DSMZ on 18 of August of 2021,
DSM 33987 deposited at DSMZ on 18 of August of 2021,
DSM 33985 deposited at DSMZ on 18 of August of 2021,
DSM 33986 deposited at DSMZ on 18 of August of 2021,
a mutant of DSM 33988 deposited at DSMZ on 18 of August of 2021,
a mutant of DSM 33987 deposited at DSMZ on 18 of August of 2021,
a mutant of DSM 33985 deposited at DSMZ on 18 of August of 2021,
a variant of DSM 33988 deposited at DSMZ on 18 of August of 2021,
a variant of DSM 33987 deposited at DSMZ on 18 of August of 2021,
a variant of DSM 33985 deposited at DSMZ on 18 of August of 2021,
a variant of DSM 33986 deposited at DSMZ on 18 of August of 2021.

15. A method for expressing a lipase from Yarrowia lipolytica in a Yarrowia lipolytica cell or Yarrowia lipolytica strain, wherein the method comprises the following steps:
expressing the lipase or mixture of lipases from Yarrowia lipolytica in a Yarrowia lipolytica cell or Yarrowia lipolytica strain in a growth medium without a carbon source selected from sugars, olive oil, tributyrin, or mixtures thereof, preferably without glucose;
wherein the lipase or mixture of lipases has a higher specificity towards the release of C₄-fatty acids from a dairy composition comprising milk fat and/or other fat if compared with the release of C₆-fatty acids, or if compared with the release of C₁₂-fatty acids or if compared with the release of C_{18:2}-fatty acids or if compared with the release of C_{18:0}-fatty acids; and/or
wherein the lipase or mixture of lipases has a higher specificity towards the release of C₄-fatty acids from a dairy composition comprising milk fat and/or other fat than SEQ ID NO: 9 or 10;
preferably wherein the Yarrowia lipolytica cell or Yarrowia lipolytica strain is a non-genetically modified Yarrowia lipolytica cell or Yarrowia lipolytica strain;
preferably wherein the lipase comprises an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4, or wherein the mixture of lipases comprises at least an amino acid sequence having at least 90%, or at least 95%, or at least 96%, or at least 97%, or at least 98%, or at least 99% or 100% identity with SEQ ID NO: 1 or SEQ ID NO: 2 or SEQ ID NO: 3 or SEQ ID NO: 4.
